(19) United States
(12) Patent Application Publication
(10) Pub. No.: US 2026/0363637 A1
(43) Pub. Date: Nov. 19, 2026

(54) X-RAY CT APPARATUS, MEDICAL INFORMATION PROCESSING APPARATUS, METHOD, AND STORAGE MEDIUM

(71) Applicant: CANON MEDICAL SYSTEMS CORPORATION, Tochigi (JP)

(72) Inventors: Shohta TAKAMI, Kamagaya-shi (JP); Fumimasa SHIGE, Kawasaki-shi (JP); Gakuto AOYAMA, Kawasaki-shi (JP)

(73) Assignee: CANON MEDICAL SYSTEMS CORPORATION, Tochigi (JP)

(21) Appl. No.: 19/571,762

(22) Filed: Mar. 19, 2026

(30) Foreign Application Priority Data
Mar. 25, 2025 (JP) 2025-050423
Feb. 9, 2026 (JP) 2026-019262

(51) Int. Cl. A61B 6/12; A61B 6/00; A61B 6/46; A61B 6/50; G06T 19/20; G16H 20/40

(52) U.S. Cl. CPC A61B 6/12; A61B 6/463; A61B 6/503; A61B 6/5217; G06T 19/20; G16H 20/40; G06T 2210/41; G06T 2219/2004

(57) ABSTRACT

An X-ray CT apparatus according to an embodiment includes processing circuitry that images a subject and acquires X-ray CT images, obtains an organ model representing an anatomical structure based on the X-ray CT images, estimates feature values after treatment based on the organ model and a plurality of installation positions in which a treatment device is installed, and causes information representing the treatment device and the installation positions to be displayed together with the organ model based on a condition on display corresponding to the feature value.

### CROSS-REFERENCE TO RELATED APPLICATIONS

This application is based upon and claims the benefit of priority from Japanese Patent Application No. 2025-050423, filed on Mar. 25, 2025; and Japanese Patent Application No. 2026-019262, filed on Feb. 9, 2026, the entire contents of which are incorporated herein by reference.

### FIELD

Embodiments described herein relate generally to an X-ray CT apparatus, a medical information processing apparatus, a method, and a storage medium.

### BACKGROUND

Various treatments provided by installing a device in the body of a subject have been known. Methods of evaluating appropriateness of each position in which a device is installed as an installation position in order to examine the position have been also proposed (Patent Literature 1-2 or the like). Users such as doctors however sometimes have difficulty in understanding a correspondence between the position in which the device is installed and the result of evaluating the position.

An object of the present invention is to provide an X-ray CT apparatus that makes it easy to grasp a correspondence between an installation position in which a device is installed and a result of evaluating the position.

An X-ray CT apparatus according to an embodiment includes a controller that images a subject and acquires X-ray CT images, an obtaining unit that obtains an organ model representing an anatomical structure based on the X-ray CT images, an estimator that estimates feature values after treatment based on the organ model and a plurality of installation positions in which a treatment device is installed, and an output unit that causes information representing the treatment device and the installation positions to be displayed together with the organ model based on a condition on display corresponding to the feature value.

According to the X-ray CT apparatus according to the embodiment, it it possible to make it easy to grasp a correspondence between an installation position in which a device is installed and a result of evaluating the installation position.

### BRIEF DESCRIPTION OF THE DRAWINGS

FIG. 1 is a block diagram illustrating an example of a medical information processing system according to a first embodiment;

FIG. 2 is a flowchart for describing a flow of a series of processes performed by a medical information processing apparatus according to the first embodiment;

FIG. 3A illustrates an example of a treatment device according to the first embodiment;

FIG. 3B illustrates an example of position vectors indicating an installation position in which the treatment device is installed according to the first embodiment;

FIG. 3C illustrates the example of the position vectors indicating the installation position in which the treatment device is installed according to the first embodiment;

FIG. 4 illustrates an example of a method of setting an installation position in which the treatment device is installed according to the first embodiment;

FIG. 5A illustrates an example of the method of setting an installation position in which the treatment device is installed according to the first embodiment;

FIG. 5B illustrates the example of the method of setting an installation position in which the treatment device is installed according to the first embodiment;

FIG. 5C illustrates an example of the method of setting an installation position in which the treatment device is installed according to the first embodiment;

FIG. 6A illustrates an example of line styles according to the first embodiment;

FIG. 6B illustrates an example of mark styles according to the first embodiment;

FIG. 7 illustrates an example of display according to the first embodiment;

FIG. 8 illustrates an example of display according to a second embodiment; and

FIG. 9 is a block diagram illustrating an example of an X-ray CT apparatus according to another embodiment.

### DETAILED DESCRIPTION

With reference to the accompanying drawings, an embodiment of a medical information processing apparatus, a method, and a program will be described in detail below.

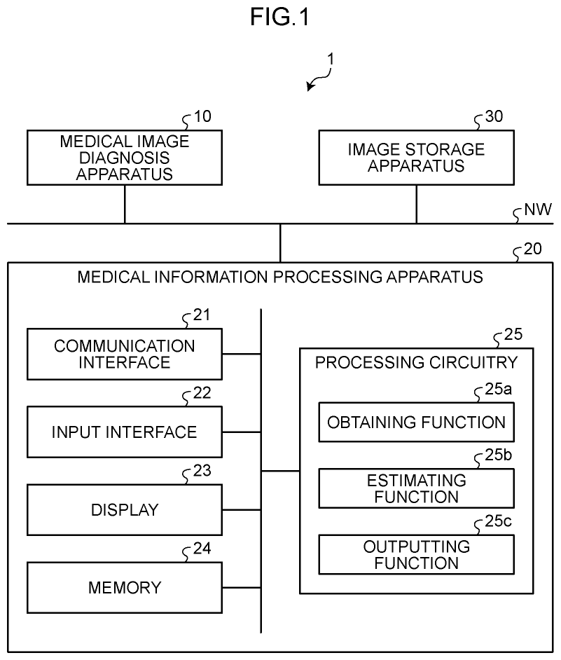

In a first embodiment, a medical information processing system 1 in FIG. 1 will be described as an example. The medical information processing system 1 includes a medical image diagnosis apparatus 10, a medical information processing apparatus 20, and an image storage apparatus 30. The medical image diagnosis apparatus 10, the medical information processing apparatus 20, and the image storage apparatus 30 are connected via a network NW such that the apparatuses can communicate with each other.

The medical image diagnosis apparatus 10 is an apparatus that images a subject and acquires medical images. The medical image diagnosis apparatus 10 is, for example, a modality apparatus such as an X-ray CT (computed tomography) apparatus, an ultrasound diagnosis apparatus, an X-ray diagnosis image, and a magnetic resonance imaging (MRI) apparatus. The medical image diagnosis apparatus 10 transmits the acquired medical images to the image storage apparatus 30 via the network NW.

The image storage apparatus 30 is an apparatus that stores various types of data acquired by the medical image diagnosis apparatus 10. For example, the image storage apparatus 30 stores various types of medical images acquired by the medical image diagnosis apparatus 10. The image storage apparatus 30 is, for example, a server of a PACS (Picture Archiving and Communication System).

Note that FIG. 1 illustrates the single medical image diagnosis apparatus 10; however, the medical information processing system 1 may include a plurality of the medical image diagnosis apparatuses 10. The medical information processing system 1 may include, for example, a plurality of types of the medical image diagnosis apparatuses 10 such as an X-ray CT apparatus and an ultrasound diagnosis apparatus.

The medical information processing apparatus 20 is an apparatus that analyzes the medical images acquired by the medical image diagnosis apparatus 10. For example, as illustrated in FIG. 1, the medical information processing apparatus 20 includes a communication interface 21, an input interface 22, a display 23, a memory 24, and processing circuitry 25.

The communication interface 21 controls transmission and communication of various types of data transmitted and received between the medical information processing apparatus 20 and another apparatus or another system that are connected via the network NW. Specifically, the communication interface 21 is connected to the processing circuitry 25 and outputs data that is received from another apparatus or another system to the processing circuitry 25 or transmits data that is output from the processing circuitry 25 to another apparatus or another system. For example, the communication interface 21 is enabled with a network card and a network adapter, a network interface controller (NIC), etc.

The input interface 22 receives various types of input operations from a user of the medical information processing apparatus 20, converts the received input operations into electric signals, and outputs the electric signals to the processing circuitry 25. For example, the input interface 22 is enabled with a mouse and a keyboard, a trackball, a switch, a button, a joystick, a touch pad via which an input operation is made by touching an operation screen of the touch pad, a touch screen obtained by integrating a display screen and a touch pad, a non-contact input circuit using an optical sensor, an audio input circuit, or the like. The input interface 22 may consist of a tablet terminal device enabling radio communication with the main unit of the medical information processing apparatus 20, or the like. The input interface 22 may be a circuit that receives an input operation from the user by motion capture. In an example, by processing signals that are obtained via a tracker and images that are acquired with respect to the user, the input interface 22 is able to receive the body motion or lines of sight of the user as an input operation. The input interface 22 is not limited to one including physical operational parts such as a mouse and a keyboard. For example, examples of the input interface 22 include an electric signal processing circuit that receives an electric signal corresponding to an input operation from an external input device provided independently of the medical information processing apparatus 20 and that outputs the electric signal to the processing circuitry 25.

The display 23 displays various types of information. For example, the display 23 displays a graphical user interface (GUI) for receiving various types of instructions, settings, etc., from the user via the input interface 22. For example, the display 23 is a liquid crystal display or a cathode ray tube (CRT) display. The display 23 may be desktop or may consist of a tablet terminal device enabling radio communication with the main unit of the medical information processing apparatus 20, or the like.

Note that the medical information processing apparatus 20 may include a projector instead of or in addition to the display 23. The projector is able to make a projection on a screen, a wall, a floor, or the like, under the control of the processing circuitry 25. In an example, the projector is able to make a projection on a given plane or an object, in a space, or the like, by projection mapping.

The memory 24 is enabled with, for example, a semiconductor memory device, such as a random access memory (RAM) or a flash memory, a hard disk, an optical disk, or the like. For example, the memory 24 stores medical images that are acquired by the medical image diagnosis apparatus 10 and various types of methods that are generated by the processing circuitry 25. The processing circuitry 25 stores a program that enables circuitry contained in the medical information processing apparatus 20 to implement its function. The memory 24 may be enabled with a server group (cloud) that is connected to the medical information processing apparatus 20 via the network NW.

The processing circuitry 25 includes an obtaining function 25a, an estimating function 25b, and an outputting function 25c. The obtaining function 25a is an example of an obtaining unit. The estimating function 25b is an example of an estimation unit. The outputting function 25c is an example of an output unit.

For example, the processing circuitry 25 reads a program corresponding to the obtaining function 25a from the memory 24 and executes the program, thereby functioning as the obtaining function 25a. Similarly, the processing circuitry 25 can function as the estimating function 25b and the outputting function 25c. Details of the obtaining function 25a, the estimating function 25b and the outputting function 25c will be described below.

In the medical information processing apparatus 20 illustrated in FIG. 1, each of the processing functions is stored in a form of a computer-executable program in the memory 24. The processing circuitry 25 is a processor that reads a program from the memory 24 and executes the program, thereby implementing the function corresponding to each program. In other words, the processing circuitry 25 having read a program includes the function corresponding to the read program.

FIG. 1 illustrates that the single processing circuitry 25 implements the obtaining function 25a, the estimating function 25b and the outputting function 25c; however, a plurality of independent processors may be combined to configure the processing circuitry 25 and the respective processors may execute the programs, thereby implementing the functions. Each of the processing functions that the processing circuitry 25 includes may be distributed or integrated to or into a single or a plurality of processing circuits as appropriate and be implemented.

The processing circuitry 25 may implement the functions using a processor of an external apparatus that is connected via the network NW. For example, the processing circuitry 25 reads the program corresponding to each of the functions from the memory 24 and executes the program and uses a server group (cloud) that is connected to the medical information processing apparatus 20 via the network NW as calculation resources, thereby implementing each of the functions illustrated in FIG. 1.

The network NW illustrated in FIG. 1 may consists of a local network closed in a facility or may be a network via the Internet. For example, the medical information processing apparatus 20 may be installed in the same facility as that where the medical image diagnosis apparatus 10 and the image storage apparatus 30 are set or may be installed in a different facility.

The entire configuration of the medical information processing system 1 including the medical image diagnosis apparatus 10, the medical information processing apparatus 20, and the image storage apparatus 30 has been described. Under the configuration, the medical information processing apparatus 20 makes it possible to make it easy to grasp a correspondence between an installation position in which a device is installed and a result of evaluating the position.

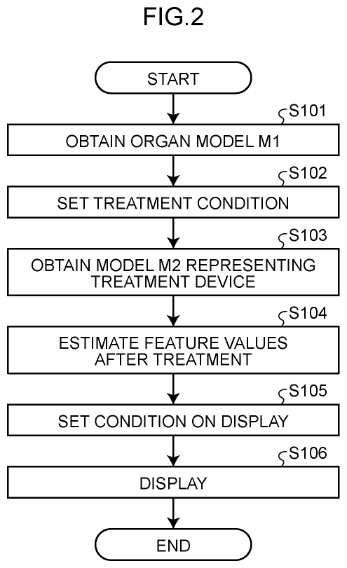

An embodiment will be described in detail along the flowchart in FIG. 2. FIG. 2 is a flowchart for describing a flow of a series of processes performed by the medical information processing apparatus 20 according to the first embodiment.

Each step in FIG. 2 is executed by the processing circuitry 25 that the medical information processing apparatus 20 includes. Specifically, step S101 and step S103 correspond to the obtaining function 25a. Step S102 and step S104 correspond to the estimating function 25b. Step S105 and step S106 correspond to the outputting function 25c.

First of all, the processing circuitry 25 obtains an organ model M1 (step S101). The organ model M1 is data expressing features of a shape of an anatomical structure to be treated. In the embodiment, for example, an example in which the organ model M1 representing a mitral valve is obtained will be described.

The organ model M1, for example, is expressed by data of a group of a given number of points forming a surface of the shape of the anatomical structure. The organ model M1 may be expressed by data of a group of a given number of points forming an area of the shape of the anatomical structure, mesh data in which the data of the group of points serves as nodes, or the like. In the embodiment, the example where the organ model M1 is mesh data will be described as an example.

The processing circuitry 25 is able to obtain the organ model M1 based on templates or the medical images acquired from the subject. The templates are organ models serving as references that are determined by reference values, experiences, etc. The medical images are acquired by the medical image diagnosis apparatus 10 illustrated in FIG. 1. Note that it is possible to obtain the organ model M1 based on only the templates as a specific example will be described below. In this case, the medical information processing system 1 need not include the medical image diagnosis apparatus 10.

When the templates are used, first of all, the templates are generated in advance for various types of anatomical structures and the templates are saved in the memory 24. The processing circuitry 25 is able to obtain a template stored in the memory 24 as the organ model M at step S101.

Alternatively, the processing circuitry 25 may use data obtained by appropriately processing a template stored in the memory 24 as the organ model M1. For example, the processing circuitry 25 modifies a template based on input information that is given by an input operation performed by the user via the input interface 22.

The input information for modifying the template may be any information as long as the information represents a change in the shape of the template. For example, the input information is obtained by dragging one of nodes contained in mesh data by an operation on a mouse, or the like, performed by the user. For example, the input information may be information indicating that a valve ring shape is enlarged about a center of gravity of the valve ring (an enlargement coefficient is determined by the distance of dragging).

When the organ model M1 is obtained based on the medical images, the processing circuitry 25 obtains the medical images acquired by the medical image diagnosis apparatus 10. For example, the medical image diagnosis apparatus 10 images the subject and acquires the medical images and then transmits the acquired images to the image storage apparatus 30. The image storage apparatus 30 manages the received medical images in a system, such as a PACS. The processing circuitry 25 is able to obtain the medical images of the subject S from the image storage apparatus 30. Alternatively, the processing circuitry 25 may obtain the medical images directly from the medical image diagnosis apparatus 10.

The type of the medical image is not limited and an X-ray CT image, an ultrasound image (echo image), an X-ray image such as angiographic image, or an MR image can be exemplified.

Various types of image processing techniques are usable as a method of obtaining the organ model M1 from the medical images. In order to obtain the organ model M, the processing circuitry 25 may use Graph Cut, Mean-Shift, or a method by deep learning (DL) such as U-net. For example, the processing circuitry 25 is able to obtain the organ model M1 using the method disclosed in Non-patent Literature 1. Alternatively, the processing circuitry 25 may obtain the organ model M1 by modifying the above-described template based on the medical images of the subject.

Next, the processing circuitry 25 sets treatment conditions (step S102). The treatment conditions include at least a plurality of installation positions in which a treatment device is installed. In the embodiment, the word “a plurality of installation positions” is described as one corresponding to a single treatment condition. For example, as described below, in the case where mitral insufficiency is treated using a clip-formed device, installation positions are set in an anterior leaflet and a posterior leaflet, respectively. Such two installation positions correspond to “a plurality of installation positions”.

At step S102, the processing circuitry 25 may set a plurality of treatment condition and set installation positions different according to every treatment condition. In other words, the processing circuitry 25 may set a plurality of installation positions in which the treatment device is installed with respect to each of the treatment conditions.

The treatment conditions may include the number of treatment devices and the type and the size of the treatment device. The treatment conditions may also include any condition that is selected or determined by a doctor on treatment. For example, the processing circuitry 25 prepares a text box and a spin button on a GUI not illustrated in the drawings and thus is able to receive selection of the number of treatment devices via an operation performed by the user using a mouse, or the like.

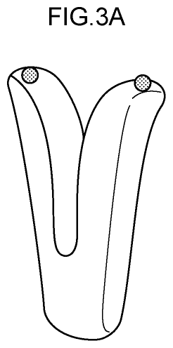

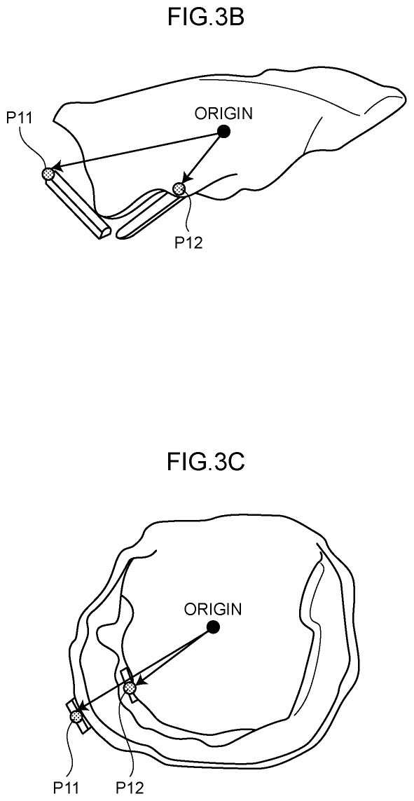

One of types of treatment devices used to treat the mitral valve is a clip-formed device like that illustrated in FIG. 3A. The clip-formed device holds the tips of the two leaves (leaflets) forming the mitral valve, thereby being able to inhibit a regurgitant flow in the mitral valve with insufficiency.

An artificial valve ring that is installed in a contour part of the mitral valve and that reinforces the valve ring has been also known as another type of treatment devices used to treat the mitral valve. A wire-formed device that is installed along coronary veins and that inhibits relaxation of the mitral valve has been also known as another type. An artificial mitral valve having a structure and a function similar to those of the mitral valve has been also known as another type. Even if a treatment device of any type is used, it is important to install the treatment device in an appropriate position.

For example, the processing circuitry 25 prepares a drop-down list, a combo box, or the like on the GUI and receives selection of a type of the treatment device via an operation on a mouse, or the like, by the user. A list of sizes corresponding to the types of treatment devices is stored in the memory 24 in advance and the processing circuitry 25 presents the list of selectable sizes to the user according to the selected type the treatment device and receives selection of a size.

Treatment device installation positions that are set as a treatment condition is positional information such as position coordinates or position vectors. For example, in the case of the clip-formed device illustrated in FIG. 3A, installation positions in which two clip tips (holders) for holding the tips of the leaflets on treatment are installed are set as a treatment condition. In other words, the installation position in which the clip-formed device is installed is at least two sets of positional information that are set with respect to the anterior leaflet and the posterior leaflet.

In an example, as illustrated in FIG. 3B and FIG. 3C, the installation positions in which the holders are installed are set as two position vectors (P11, P12) heading from a given origin toward centers of upper ends of the holders of the treatment device. Note that both FIG. 3B and FIG. 3C illustrates the mitral valve using 3D models and have different directions of display. The 3D models are an example of the organ model M1. The position vector P11 and the position vector P12 are set, for example, in a way that the user selects positions of the anterior leaflet and the posterior leaflet on a mesh of the mitral valve on the GUI on which the mesh is displayed with a mouse, or the like.

Note that FIG. 3B and FIG. 3C only illustrate the position vector P11 and the position vector P12 representing the installation positions in which the two holders are installed; however, a vector representing an orientation of the treatment device may be further set. For example, the processing circuitry 25 further sets a unit vector representing a direction of the treatment device illustrated in FIG. 3A in a longitudinal direction as the installation position in which the treatment device is installed. The unit vector is set, for example, in a way that the user rotates a single vector on the GUI on which the single vector is displayed with a mouse, or the like.

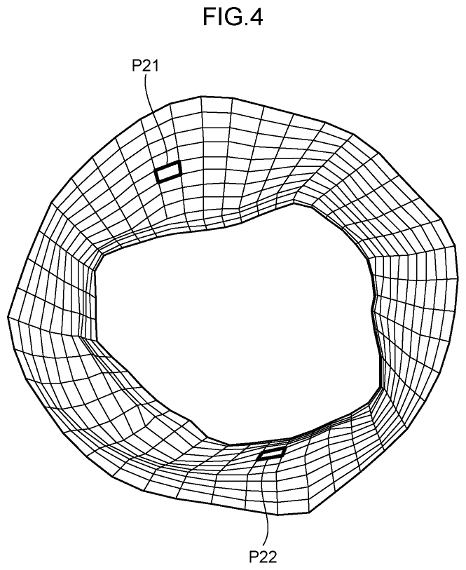

Using FIG. 4, another example with respect to the method of setting an installation position in which the treatment device is installed will be described. FIG. 4 illustrates an example of the method of setting an installation position in which the treatment device is installed according to the first embodiment.

As illustrated in FIG. 4, the organ model M1 can be expressed by mesh data that is a set of elements in which nodes serve as vertices. For example, element numbers are assigned to the respective elements of the mesh data in advance and the user selects two of the element numbers. For example, the user inputs the element numbers assigned to an element P21 and an element P22 to a text box or the like on the GUI. In this case, the processing circuitry 25 is able to obtain sets of coordinates of the respective centers of the element P21 and the elements P22 and position vectors expressing the sets of coordinates of the centers as the installation position in which the treatment device is installed.

The processing circuitry 25 may obtain the installation position in which the treatment device is installed not based on the elements of the mesh data but based on the nodes of the mesh data. For example, node numbers are assigned to the respective nodes of the mesh data and the user inputs two node numbers to a text box or the like. The processing circuitry 25 thus is able to obtain sets of coordinates of the positions of the two nodes corresponding to the input node numbers and position vectors expressing the sets of coordinates of the positions as the installation position in which the treatment device is installed.

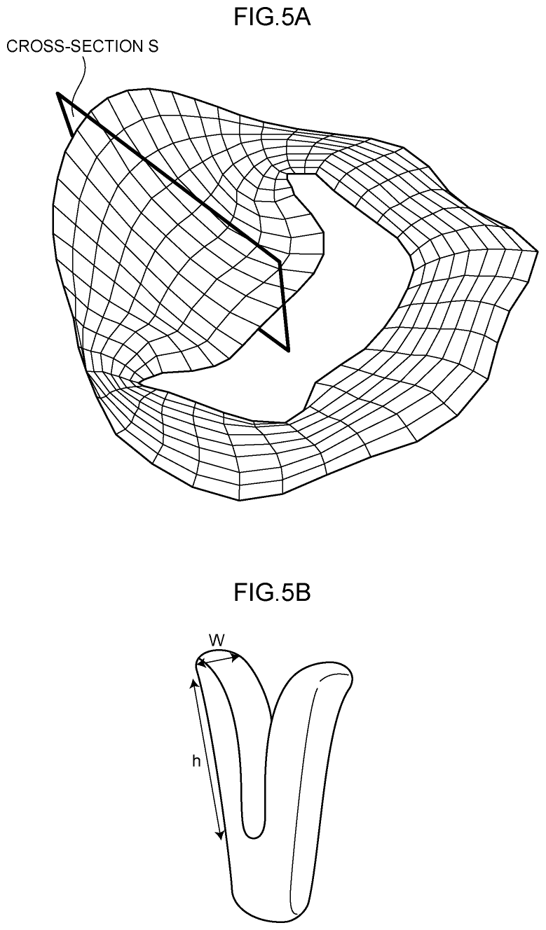

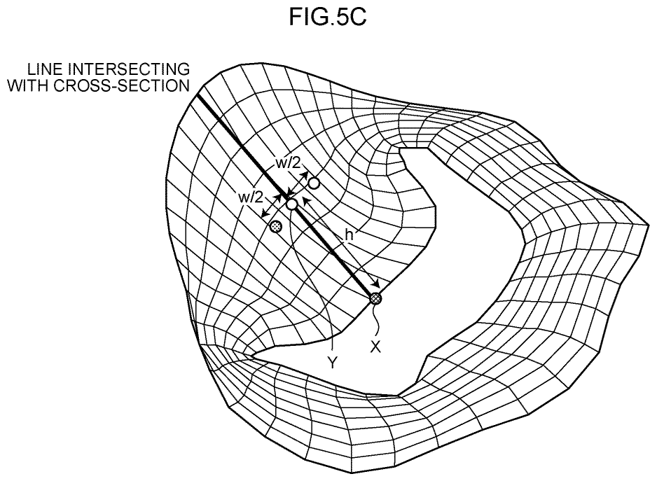

Another example with respect to the method of setting an installation position in which the treatment device is installed will be described using FIGS. 5A, 5B, and 5C. FIGS. 5A, 5B, and 5C illustrate an example of the method of setting an installation position in which the treatment device is installed according to the first embodiment.

FIG. 5A illustrates mesh data that is the organ model M1 and a cross-section S. The cross-section S corresponds to the installation position in which the treatment device is installed. For example, the processing circuitry 25 obtains sets of coordinates of positions of tree points in the cross-section S and position vectors as the installation position in which the treatment device is installed.

For example, the processing circuitry 25 first of all calculates an intersection between the cross-section S and an orifice of the anterior leaflet (referred to as point X below). The point X corresponds to a center point of a lower end of the holder of the treatment device. Next, the processing circuitry 25 calculates a point in a distance of “h” illustrated in FIG. 5B (referred to as a point Y) from the point X along a section obtained by cutting the anterior leaflet on the cross-section S. The point Y corresponds to a center point of an upper end of the holder of the treatment device.

Next, the processing circuitry 25 obtains a cross-section formed of a normal vector of the cross-section S and a normal vector of a cross-section approximately horizontal to part of or all the valve ring (referred to as a perpendicular cross-section below). The processing circuitry 25 associates two points in a distance that is half “w” illustrated in FIG. 5B (that is, w/2) from the point Y along a section obtained by cutting the anterior leaflet on the perpendicular cross-section with an upper-end and right and left end points of the holder of the treatment device in the anterior leaflet.

In other words, the holder of the tip of the device has a certain width (w) and, while representing the width by the interval between the two end points, the processing circuitry 25 sets the width as the installation position in which the holder is installed. As for the posterior leaflet, similarly, the processing circuitry 25 sets two points as the installation position in which the holder is installed. Note that, not using the two points that are set by the above-described method as the installation position directly, the processing circuitry 25 may obtain two nodes that are close to the two points that are set by the above-described method out of the nodes in the mesh data as the installation position.

The method illustrated in FIGS. 5A, 5B and 5C enables the user to adjust the installation position in which the treatment device is installed by adjusting a position and an angle of the cross-section S illustrated in FIG. 5A. For example, on the GUI on which the mesh data representing the mitral valve and the cross-section S, the processing circuitry 25 receives an operation of moving and rotating the cross-section S with respect to the mesh data or moving and rotating the mesh data with respect to the cross-section S from the user, thereby obtaining the installation position in which the treatment device is installed.

Next, the processing circuitry 25 obtains a model M2 representing the treatment device (step S103). The model M2 is an example of information representing the treatment device. The model M2 is also referred to as a device model. For example, the processing circuitry 25 obtains a model of the clip-shaped device illustrated in FIG. 3A as the model M2.

The model M2 preferably expresses the treatment condition that is set at step S102 and need not express a detailed shape of the treatment device. For example, in treatment using the clip-formed device, the two sets of positional information that are set with respect to the anterior leaflet and the posterior leaflet are set as the installation position in which the treatment device is installed. In this case, the processing circuitry 25 is able to obtain a line segment connecting the two points that are set as the installation position, a figure in any shape passing the two points, a cross-section passing the two points, or the like as the model M2.

For example, a given line segment model is saved in the memory 24 previously. For example, the processing circuitry 25 deforms the line segment model that is read from the memory 24 according to the set two sets of positional information. For example, the processing circuitry 25 obtains the two position vectors illustrated in FIG. 3B and the like and makes a deformation such that the end points of a line segment are at sets of coordinates indicated by the respective position vectors, thereby obtaining the model M2.

The mode of the model M2 may be changed according to the various types of treatment conditions that are set at step S102. For example, a thickness and a line style of the line segment, a line color, a permeation rate, and presence or absence of end points are taken as examples of the mode of the model M2 that is a line segment. Note that, when a plurality of treatment conditions are set at step S102, the processing circuitry 25 obtains the model M2 according to each of the treatment conditions.

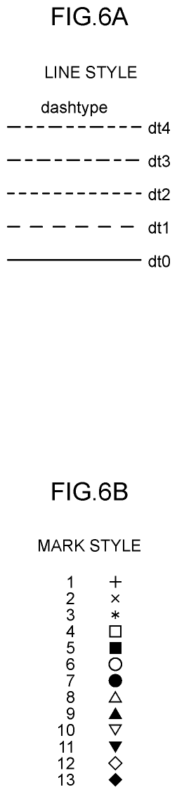

Examples of the line style are illustrated in FIG. 6A. For example, an example of the line style is a solid line presented as “dt0”. Other examples of the line style are dotted lines having equal spaces and presented as “dt1” and “dt2”. Other examples of the line style are dashed lines of various decorations presented as “dt3” and “dt4”. Needless to say, FIG. 6A illustrates examples and various modifications can be made in the spaces and decorations. When there are end points, mark tyles like those illustrated in FIG. 6B are also included in examples of the mode of the model M2.

For example, the thickness of a line segment and the mark style that are obtained as the model M2 may be changed according to the size of the treatment device that is set at step S102. In other words, the processing circuitry 25 may set a condition on display of information representing the treatment device. The processing circuitry 25 sets a condition on display such that, the larger the size of the treatment device is, the more information representing the treatment device is enhanced. For example, the larger the size of the treatment device is, the more the thickness of the line segment is set larger.

For example, the thickness of the line segment may be changed according to an angle of installation of the treatment device. In other words, the processing circuitry 25 may set a condition on display of the information representing the treatment device according to the angle of installation of the treatment device. In this case, the processing circuitry 25 sets the condition on display such that, the larger the angle of installation of the treatment device is, the more the information representing the treatment device is enhanced. For example, the processing circuitry 25 obtains, as the angle of installation of the treatment device, an angle formed by the unit vector representing an orientation of the clip-formed device illustrated in FIG. 3A in the longitudinal direction and the normal vector of the cross-section approximately horizontal to part of or all the valve ring. The processing circuitry 25 then changes the thickness of the line segment and the mark style such that the thickness increases as the angle increases. Needless to say, the processing circuitry 25 may set a condition on display of the information representing the treatment device according to both the size of the treatment device and the angle of installation of the treatment device.

When a cross-section is obtained as the model M2, a given cross-sectional model is saved in the memory 24 previously. For example, the processing circuitry 25 deforms the cross-sectional model that is read from the memory 24 according to two sets of positional information that are set as the installation position in which the treatment device is installed.

For example, the processing circuitry 25 obtains the two position vectors illustrated in FIG. 3B or the like and makes a deformation such that end points of one side of the cross-section are at sets of coordinates indicated by the respective position vectors. The processing circuitry 25 obtains the above-described angle of installation of the treatment device and rotates the cross-section by the angle of installation about the line segment connecting the two position vectors and serving as an axis, thereby obtaining the model M2. Note that the cross-sectional model that is saved previously in the memory 24 may be any model as long as the height of the model is sufficiently small with respect to the size of the valve. The thickness and the height of the cross section may be constant or may be changed according to the size of the device that is set at step S102, or the like.

Note that the example where the model M2 is obtained by deforming the line segment model or the cross-sectional model that is saved previously in the memory 24 has been described; however, embodiments are not limited to this. For example, the processing circuitry 25 may obtain the line segment model or the cross-sectional model that is saved previously in the memory 24 as the model M2 directly without deformation.

For example, the processing circuitry 25 may obtain a line segment model or a cross-sectional model that is set by a user operation, or the like, as the model M2. For example, the processing circuitry 25 may obtain the cross-section S illustrated in FIG. 5A as the model M2. Also in this case, as described above, the thickness and the height of the cross-section may be constant or may be changed according to the size of the device that is set at step S102, or the like.

Next, the processing circuitry 25 estimates feature values after treatment (step S104). The feature values after treatment are feature values according to a condition of the subject in the case where treatment is provided under the treatment condition that is set at step S102. When a plurality of treatment conditions are set at step S102, the processing circuitry 25 estimates a feature value with respect to each of the treatment conditions.

The feature values after treatment vary according to the treatment condition. For example, the feature values after treatment are indices reflecting any one or both of an effect of and a response to a treatment on the subject and a complication. For example, when mitral insufficiency is treated using the clip-formed device illustrated in FIG. 3A, the processing circuitry 25 estimates an index such as a regurgitant orifice area and a regurgitant flow or an amount change in the index before and after treatment as the feature values after treatment.

The processing circuitry 25 may estimate a composite index that is calculated based on a plurality of indices as the feature values after treatment. The composite index may be an index that is clinically used such as “MR grade” or may be an index that is freely defined. For example, the processing circuitry 25 may estimate, as the feature values after treatment, an index that is calculated according to a definition of “(regurgitant orifice area)/0.40 [cm²]+(regurgitant flow)/60 [ml])”. Various modifications can be made with respect to the method of estimating a feature value. For example, the processing circuitry 25 may estimate a feature value by the method disclosed in Non-patent Literature 2.

Next, the processing circuitry 25 estimates a condition on display (step S105). Specifically, at step S106 described below, the processing circuitry 25 causes the model M2obtained at step S103 and the installation positions that are set at step S102 to be displayed together with the organ model M1. Prior to the display, the processing circuitry 25 sets a condition on display of the model M2 based on the feature value that is estimated at step S104.

For example, when the model M2 is a line segment, the processing circuitry 25 can set a thickness and a line style of the line segment, a line color, a permeation rate, presence or absence of end points, or a mark style of end points as a condition on display of the model M2. Note that, in the case where the mode of the line segment model is changed according to the size of the treatment device, or the like, when obtaining the model M2 at step S103, the processing circuitry 25 sets a condition on display with respect to an item different from that of the changed mode. For example, the processing circuitry 25 changes the thickness of the line segment model according to the size of the treatment device and thus obtains the model M2 at step S103 and sets a permeation rate of the model M2 based on the estimated feature value at step S105.

For example, the processing circuitry 25 sets a condition on display of the model M2 such that, the larger a postoperative regurgitant flow is, the more visibility lowers. For example, the processing circuitry 25 estimates a regurgitant orifice area at step S104. The larger the regurgitant orifice area is, the more the processing circuitry 25 increases the permeation rate of the line segment or thins the line segment. For example, the processing circuitry 25 estimates a regurgitant flow at step S104. The larger the regurgitant flow is, the more the processing circuitry 25 increases the permeation rate of the line segment or thins the line segment. In other words, the processing circuitry 25 may estimate a regurgitant flow after treatment in the mitral valve and may set a condition on display of the information representing the treatment device such that, the larger the regurgitant flow is, the more the information representing the treatment device is in a mode of display different from enhanced display. That is, the processing circuitry 25 may estimate a prognostic state corresponding to the installation position in which the treatment device is installed as a feature value and may set a condition on display of the information representing the treatment device such that, the better the prognostic state is, the more the information representing the treatment device is displayed in an enhanced manner. Here, it is possible to, using a plurality of feature values that are estimated according to the installation positions in which the treatment device is installed, make a comparison on the prognostic states corresponding to the installation positions in which the treatment device is installed. It is thus possible to specify an installation position enabling a better prognostic state.

For example, the processing circuitry 25 sets, for the permeation rate of the model M2, a value obtained by normalizing the feature value that is estimated at step S104. For example, the processing circuitry 25 estimates a regurgitant orifice area at step S104, divides the estimated regurgitant orifice area by “0.40 [cm²]”, and sets, for the permeation rate, a value obtained by clipping in which “0” is a lower limit and “1” is an upper limit. Note that, in the case where a plurality of treatment conditions are set, the processing circuitry 25 estimates a regurgitant orifice area according to each of the treatment conditions and executes setting of a permeation rate based on the regurgitant orifice area with respect to each of the treatment conditions.

In another example, in the case where a plurality of treatment conditions are set, the processing circuitry 25 estimates a regurgitant orifice area according to each of the treatment conditions at step S104 and specifies a maximum value and a minimum value among the estimated regurgitant orifice areas. The processing circuitry 25 may set, for the permeation rate, a value obtained by normalizing the regurgitant orifice area using the specified maximum value and the minimum value.

In another example, the processing circuitry 25 estimates a regurgitant orifice area at step S104 and further estimates “MR grade” based on the regurgitant orifice area. The processing circuitry 25 then sets a permeation rate of the model M2 according to “MR grade”. For example, the processing circuitry 25 sets a permeation rate of “0%” when “MR grade” is “mild”, sets a permeation rate of “50%” when “MR grade” is “moderate”, and sets a permeation rate of “90%” when “MR grade” is “severe”.

In another example, the processing circuitry 25 estimates a regurgitant flow with respect to each treatment condition at step S104 and sets a permeation rate of the model M2 according to an order in which the regurgitant flows are rearranged in an ascending order. For example, the processing circuitry 25 makes a change in the permeation rate between the top five smaller regurgitant flows and other regurgitant flows. For example, the processing circuitry 25 sets “0%” with respect to the top five and sets “80%” with respect to the 6th and lower. In other words, the processing circuitry 25 causes the information representing the treatment device and a plurality of installation positions that correspond to a give number of feature values of larger postoperative regurgitant flows among the feature values that are estimated with respect to the treatment conditions, respectively, to be displayed. Similarly, the processing circuitry 25 may cause the information representing the treatment device and a plurality of installation positions that correspond to a given ratio of feature values of larger postoperative regurgitant flows among the feature values that are estimated with respect to the treatment conditions, respectively, to be displayed. Similarly, the processing circuitry 25 may cause the information representing the treatment device and the installation positions that correspond to a given number of or a given ratio of feature values of smaller postoperative orifice areas among the feature values that are estimated with respect to the treatment conditions, respectively, to be displayed.

In another example, the processing circuitry 25 estimates a regurgitant flow with respect to each treatment condition at step S104 and extracts estimated regurgitant flows with respect to a treatment condition that the treatment device is in a specific size. The processing circuitry 25 then rearranges the extracted regurgitant flows in an ascending order and sets a permeation rate of the model M2 according to the order after rearrangement.

In another example, the processing circuitry 25 estimates a regurgitant flow with respect to each treatment condition at step S104. The processing circuitry 25 also obtains the above-described angle of installation of the treatment device with respect to each treatment condition and extracts estimated regurgitant flows with respect to a treatment condition that the angle of installation is within a given range. The processing circuitry 25 then rearranges the extracted regurgitant flows in an ascending order and sets a permeation rate of the model M2 according to the order after rearrangement. In other words, the processing circuitry 25 may set the condition on display according to at least one of the size of the treatment device and the angle of installation of the treatment device. For example, the processing circuitry 25 sets a condition on display such that, the larger the size is, or the larger the angle of installation is, the more the information representing the treatment device is enhanced.

The example where a permeation rate is set as the condition on display of the model M2 has been described; however, the embodiment is not limited to this. For example, the processing circuitry 25 may set a color of the model M2 in addition to a permeation rate. When two conditions of a permeation rate and a color are set as the condition on display, a method of setting a permeation rate and a method of setting a color are preferably different. For example, the processing circuitry 25 sets a permeation rate of the model M2 based on the regurgitant orifice areas estimated at step S104 and sets a color of the model M2 based on the regurgitant flows estimated at step S104.

The processing circuitry 25 then makes a display based on the condition on display that is set according to the feature values after treatment (step S106). Specifically, based on the condition on display that is set, the processing circuitry 25 causes the model M2 that is obtained at step S103 and the installation positions that are set at step S102 to be displayed together with the organ model M1.

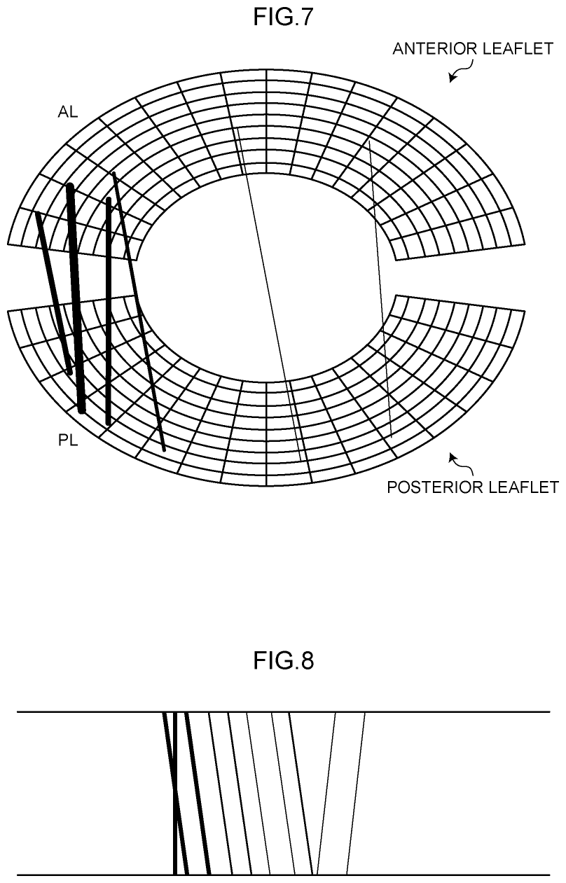

For example, as illustrated in FIG. 7, the processing circuitry 25 causes mesh data to be displayed as the organ model M1. Furthermore, under the condition on display that is set at step S105, the processing circuitry 25 causes the model M2 to be displayed according to the installation positions that are set at step S102. FIG. 7 illustrates an example of display according to the first embodiment.

Specifically, according to FIG. 7, the installation positions are set in the anterior leaflet and the posterior leaflet, respectively, as the installation position in which the treatment device is installed. In FIG. 7, the model M2 is a line segment and is displayed in a position where the installation position that is set in the anterior leaflet and the installation position that is set in the posterior leaflet are connected. In other words, the model M2 is displayed according to the two installation positions that are set in the anterior leaf and the posterior leaflet. In FIG. 7, six models M2 are displayed according to six treatment conditions that are set. In other words, six treatment conditions are set in FIG. 7 and the processing circuitry 25 causes the information representing the treatment device and the six installation positions. Note that the processing circuitry 25 may display only the single model M2. In other words, the processing circuitry 25 may set only one treatment condition and cause the information representing the treatment device and the installation positions that correspond to the single treatment condition to be displayed.

Note that the displayed model M2 may be all the models that are obtained at step S103 or only a model that satisfies a specific condition among the models M2 obtained at step S103.

For example, the processing circuitry 25 sets a size of the treatment device as the specific condition. In this case, the processing circuitry 25 causes only a model M2 that meets the specific condition in the size of the treatment device that is set at step S102 among the models M2 obtained at step S103 to be displayed. It is possible to set the specific condition, for example, in a way that a drop-down list, a combo box, or the like is prepared on the GUI and a selecting or inputting operation is received from the user. A list of sizes corresponding to each treatment device may be stored in the memory 24 in advance and a list of sizes selectable according to the type of the treatment device that is selected at step S102 may be changed. Alternatively, the displayed model M2 may be added with information on the device size and the device size may be obtained from information on the model of the treatment device that is selected using the GUI, or the like.

For example, the processing circuitry 25 may obtain the above-described angle of installation of the treatment device and, using the angle of installation as a specific condition, cause only the model M2 satisfying the specific condition to be displayed. The specific condition may be set, for example, in a way that the processing circuitry 25 prepares two text boxes, or the like, on the GUI and an operation of inputting an angle range is received from the user. Alternatively, information on the above-described angle may be added to the displayed model M2, the above-described angle may be obtained from the information of the model of the treatment device that is selected using the GUI or the like, and an angle range (for example, “±10°”) may be set based on the angle.

The processing circuitry 25, for example, may display only a condition with the smallest feature value calculated at step S104 among a plurality of treatment conditions that are set at step S102 and that are the same in two position vectors indicating an installation position in which the treatment device is installed and are different in the size of the device. Alternatively, or furthermore, the processing circuitry 25 may display only a condition with the smallest feature value calculated at step S104 among a plurality of conditions that are the same in two position vectors indicating an installation position in which the treatment device is installed and are different in the above-described angle of installation of the treatment device.

As described above, the obtaining function 25a according to the first embodiment obtains an organ model M1 representing an anatomical structure. The estimating function 25b estimates feature values after treatment based on the organ model and a plurality of installation positions in which a treatment device is installed. Based on a condition on display corresponding to the feature value, the outputting function 25c causes a model M2 representing the treatment device and the installation positions to be displayed together with the organ model M1. This enables the medical information processing apparatus 20 to make it easy to grasp a correspondence between the installation position in which the device is installed and a result of evaluating the position.

Note that, estimating feature values with respect to respective positions on the organ model, assigning colors to the feature values, and making a color map display are assumed as another method of displaying the correspondence between the installation position in which the device is installed and the result of evaluating the position. The method however is applicable to only the case where a single installation position is set as the treatment condition. On the other hand, the medical information processing apparatus 20 according to the first embodiment is able to deal with the case where a plurality of installation positions are set as the treatment condition as in the case where mitral insufficiency is treated by holding in the respective positions of the anterior leaflet and the posterior leaflet and is able present an evaluation on the installation positions.

In the above-described first embodiment, the example where mitral insufficiency is treated with the clip-formed device illustrated in FIG. 3A has been mainly described. On the other hand, in a second embodiment, an example in which percutaneous coronary intervention (PCI) treatment on coronary artery stenosis is provided using a stent that is made indwell in a blood vessel will be described.

The medical information processing system 1 according to the second embodiment has a configuration similar to that of the medical information processing system 1 illustrated in FIG. 1 and is different in part of the processes performed by the obtaining function 25a, the estimating function 25b, and the outputting function 25c. As for the aspects described in the first embodiment, the same reference numerals as those in FIG. 1 are assigned and description thereof will be omitted.

For example, at step S101, the processing circuitry 25 obtains the organ model M1, for example, like that illustrated in FIG. 8. FIG. 8 illustrates a cross-sectional view of the organ model M1 taken along a core of a coronary artery. Needless to say, FIG. 8 is a view simplified for illustration; however, the organ model M1 of the coronary artery represents a tubular structure like that in FIG. 8.

The processing circuitry 25 sets an installation position in which a stent that is a treatment device is installed as one treatment condition that is set at step S102. For example, the processing circuitry 25 obtains positions in two points where a distal end of the stent makes contact with the coronary artery. In other words, when making the stent indwell on the cross-section of the coronary artery like that illustrated in FIG. 8 is assumed, the distal end of the stent makes contact in two points with an upper wall surface and a lower wall surface of the coronary artery. The processing circuitry 25 sets these two points as a plurality of installation positions in which the treatment device is installed.

At step S103, the processing circuitry 25 obtains the model M2 representing the stent. For example, the processing circuitry 25 obtains a line segment connecting two points that are a point indicating an upper part of the distal end of the stent and a point indicating a lower part of the distal end of the stent. The processing circuitry 25 may set a thickness and a line style of the line segment, a line color, a permeation rate, presence or absence of end points, or a mark style according to a type and size of the stent.

At step S104, the processing circuitry 25 estimates feature values after treatment. For example, the processing circuitry 25 estimates a fractional flow reserve (FFR) value serving as a postoperative index. At step S105, the processing circuitry 25 sets a condition on display of the model M2 (such as a permeation rate) according to the FFR value. As illustrated in FIG. 8, the processing circuitry 25 then causes the model M2 representing the stent and the installation positions in which the stent is installed to be displayed together with the organ model M1. Note that FIG. 8 illustrates, as an example, ten kinds of display of the model M2 representing the stent and the installation positions in which the stent is installed according to the treatment condition. Note that, in FIG. 8, the thickness of the model M2 presented by a line segment is changed according to the feature value such as the FFR value.

In addition to the above-described first and second embodiments, embodiments added with various modifications may be carried out.

For example, in the above-described first and second embodiments, the mitral valve is exemplified as the anatomical structure and is described; however, embodiments are not limited to this. For example, in the above-described embodiment, for example, a biological organ may be any one, such as an aortic valve, a tricuspid valve, a left atrial appendage, a coronary artery, or a cerebral artery, as long as the treatment in which the treatment device is made indwell in part of whole the structure is performed on the biological organ.

Alternatively, the above-described embodiment may target at anatomical structures excluding biological organs. The anatomical structures excluding biological organs are, for example, substances attached to the biological organs described above. Calcification, plaque, a wart, a treatment device that is already indwelling, etc., can be exemplified as the substances.

In the above-described first and second embodiments, the example where a template is modified based on input information from the user in the case where the organ model M1 is obtained by modifying a template has been described. Embodiments however are not limited to this. For example, the processing circuitry 25 may modify the template according to patient information and examination information on the subject, or the like.

For example, the processing circuitry 25 saves data of a template of an organ model previously in the memory 24 and reads the data at the start of step S101. The processing circuitry 25 further obtains an electronic medical record of a subject to be treated, obtains patient information from the electronic medical record, and deforms the template based on the patient information, thereby obtaining the organ model M1.

The patient information may be any information as long as the information expresses a physical feature of the subject to be treated. A weight, a height, a gender, a race, a name of a disease, etc., can be taken as specific examples of the patient information. For example, when the gender is female, the template is deformed by multiplying the shape of the template by a coefficient that is set previously. For example, when the weight or the height exceeds a threshold that is set previously, the template is deformed by multiplying the shape of the template by a coefficient that is set previously.

In another example, the processing circuitry 25 saves data of a template of an organ model previously in the memory 24 and reads the data at the start of step S101. The processing circuitry 25 further obtains an electronic medical record of a subject to be treated, obtains measured values relating to a disease from the electronic medical record, and deforms the template based on the measured values, thereby obtaining the organ model M1.

The measured values are, for example, a regurgitant flow, a regurgitant orifice area, and a vena contracta. For example, the template is deformed by multiplying the shape of the template by a coefficient proportional to the magnitude of the regurgitant flow. For example, when the regurgitant orifice area exceeds a threshold that is set previously, the template is deformed by enlarging the shape of a valve ring by multiplication by a coefficient that is set previously.

The patient information and the measured values can be obtained from a DICOM (Digital Imaging and Communications in Medicine) tag ancillary to a medical image obtained from the medical image diagnosis apparatus 10 or the image storage apparatus 30. The patient information and the measured values may be obtained by an input made by the user.

In the above-described first and second embodiments, the examples in which the image processing techniques are used to obtain the organ model M1 from medical images are described. Embodiments however are not limited to this.

For example, the processing circuitry 25 may obtain part of a model from medical images, retrieve a template that is saved in a storage previously for the remaining part, and combine the template with the model obtained from the medical images, thereby obtaining the organ model M1. For example, the processing circuitry 25 may obtain the valve ring of the mitral valve from medical images by various types of image processing techniques, retrieve a template of a plurality of chorda tendineae and a papillary muscle that is saved in the storage previously, and combine the template with the model based on information on a positional relationship between each of the chorda tendineae and the mitral valve that is saved in the template, thereby obtaining the organ model M1. Note that the information on the positional relationship between each of the chorda tendineae and the mitral valve is, for example, information like one indicating that the tip of the chorda tendinea is a center of gravity of the anterior leaflet.

The processing circuitry 25 may determine whether or not to display the model M2 before setting a condition on display at step S105 with respect to the model M2. For example, the processing circuitry 25 may make a setting to display only the models M2 corresponding to the treatment conditions of the top five of a plurality of regurgitant orifice areas that are estimated with respect to a plurality of treatment conditions, respectively, and make a setting to display only the models M2 corresponding to the treatment conditions of the top 5% of the regurgitant orifice areas.

In the first and second embodiment, the angle formed by the unit vector indicating the orientation of the clip-formed device illustrated in FIG. 3A in the longitudinal direction and the normal vector of the cross-section approximately horizontal to part of or all the valve ring is taken as an example of the angle of installation in which the treatment device is installed and is described. Embodiments however are not limited to this.

For example, even when a treatment device other than the clip-formed device is used, similarly, it is possible to set a unit vector according to a feature of the treatment device and define an angle of installation in which the treatment device is installed. Even when an anatomical structure other than the mitral valve is targeted, similarly, it is possible to set a normal vector according to a feature of the anatomical structure and define an angle of installation in which the treatment device is installed.

A plurality of treatment devices may be set in the organ (mitral valve). Providing treatment using at least two clip-formed devices is assumed. The clip-formed devices make it possible to hold each of the tips of the two leaves (leaflets) forming the mitral valve.

The processing circuitry 25 estimates feature values after treatment based on the organ model and a plurality of installation positions corresponding to the treatment devices. Based on a condition on display corresponding to the feature value, the processing circuitry 25 then causes information representing the treatment devices and the installation positions corresponding to the treatment devices to be displayed together with the organ model.

In the above-described embodiments, the medical image diagnosis apparatus 10 and the medical information processing apparatus 20 are described as different configurations; however, the medical image diagnosis apparatus 10 and the medical information processing apparatus 20 may be integrated. For example, the medical information processing apparatus 20 may be a console device that the medical image diagnosis apparatus 10 includes. In this case, the medical information processing apparatus 20 is able to obtain medical images by capturing images of a subject.

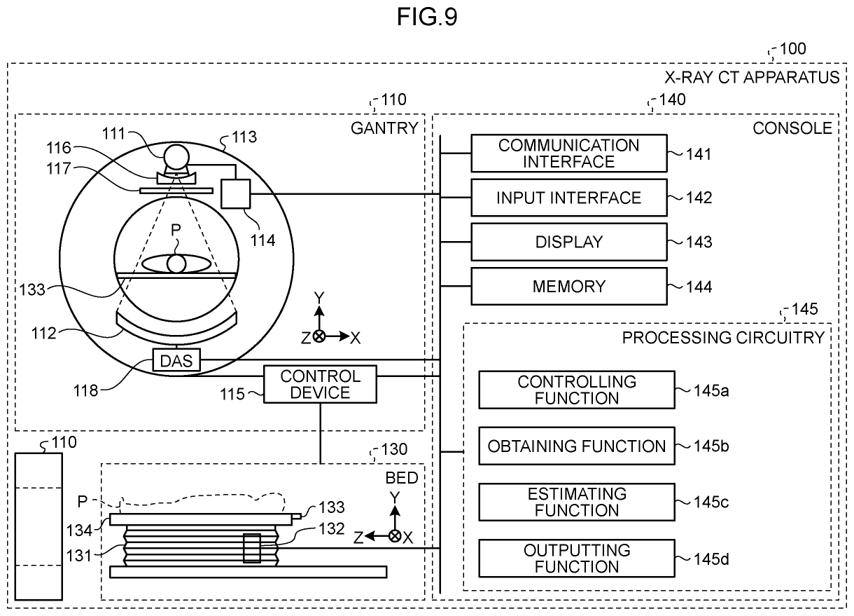

FIG. 9 illustrates an X-ray CT apparatus 100 as an example of the medical image diagnosis apparatus 10 and an example in which the same process as that executed by the medical information processing apparatus 20 is executed in the X-ray CT apparatus 100 will be described. FIG. 9 is a block diagram illustrating an example of the X-ray CT apparatus 100 according to another embodiment. For example, the X-ray CT apparatus 100 includes a gantry 110, a bed 130, and a console 140.

In FIG. 9, a rotational axis of a rotation frame 113 in a non-tilt state or a longitudinal direction of a tabletop 133 of the bed 130 is Z-axis direction. The Z-axis direction corresponds to a direction of a body axis of a subject P who is laid on the tabletop 133. An axial direction that is orthogonal to the Z-axis direction and that is parallel to a floor surface is an X-axis direction. An axial direction that is orthogonal to the Z-axis direction and that is perpendicular to the floor surface is a Y-axis direction. Note that FIG. 2 is a drawing of the gantry 110 in multiple directions for description and illustrates the case where the X-ray CT apparatus 100 includes the single gantry 110.

The gantry 110 includes an X-ray tube 111, an X-ray detector 112, the rotation frame 113, an X-ray high-voltage device 114, a control device 115, a wedge 116, a collimator 117, and a data acquisition system (DAS) 118.

The X-ray tube 111 is a vacuum tube including a cathode (filament) that generates thermoelectrons and an anode (target) that generates X-rays in response to collision of the thermoelectrons. The X-ray tube 111 applies thermoelectrons from the cathode to the anode in response to application of a high voltage from the X-ray high voltage device 114, thereby generating X-rays to be applied to the subject P. The X-ray tube is an example of an X-ray generator.

Note that hardware that generates X-rays is not limited to the X-ray tube 111. For example, instead of the X-ray tube 111, X-rays may be generated using a fifth-generation system including a focus coil that converges electron beams generated by an electron gun, a deflection coil that causes electromagnetic deflection, and a target ring in which collision of deflected electron beams surrounding half around the subject P causes X-rays. In other words, the X-ray generator may be configured using a focus coil, a deflection coil, and a target ring.

The X-ray detector 112 detects X-rays having been applied from the X-ray tube 111 and having passed through the subject P and outputs a signal corresponding toa a detected x-ray dosage to the DAS 118. The X-ray detector 112, for example, includes a plurality of detection element rows in which a plurality of detection elements are arrayed in a channel direction along an arc about a focal point of the X-ray tube 111. The X-ray detector 112, for example, has a configuration in which a plurality of detection element rows in which a plurality of detection elements are arrayed in the channel direction are arrayed in the row direction (slice direction or row direction). The X-ray detector 112 is an example of an X-ray detector.

For example, the X-ray detector 112 is an indirect conversion detector including a grid, a scintillator array, and an optical sensor array. The scintillator array includes a plurality of scintillators. The scintillator includes a scintillator crystal that outputs light of a photon quantity corresponding to a dosage of incident X-rays. The grid is arranged on a face of the scintillator array on a side of incidence of X-rays and includes an X-ray shield that absorbs scattering X-rays. Note that the grid is sometimes referred to as a collimator (one-dimensional collimator or a two-dimensional collimator). An optical sensor array has a function of conversion into an electric signal corresponding to the amount of light from the scintillator and includes an optical sensor such as a photodiode. Note that the X-ray detector 112 may be a direct conversion detector including a semiconductor device that converts incident X-rays into electric signals. The X-ray detector 112 may be a photon counting detector that outputs a signal by which an energy value of an incident X-ray photon can be measured.

The rotation frame 113 is an annular frame that supports the X-ray tube 111 and the X-ray detector 112 such that the X-ray tube 111 and the X-ray detector 112 are opposed to each other and that is caused by the control device 115 to rotate the X-ray tube 111 and the X-ray detector 112. For example, the rotation frame 113 is a cast made of aluminum. The rotation frame 113 is also able to further support, in addition to the X-ray tube 111 and the X-ray detector 112, the X-ray high-voltage device 114, the wedge 116, the collimator 117, the DAS 118, etc. Furthermore, the rotation frame 113 is capable of further supporting various structures not illustrated in FIG. 1. The rotation frame 113 and parts that rotate together with the rotation frame 113 in the gantry 110 are also referred to as a rotation unit.

The X-ray high-voltage device 114 includes electric circuits such as a transformer and a rectifier and includes a high-voltage generation device that generates a high voltage to be applied to the X-ray tube 111 and an X-ray control device that performs control on an output voltage corresponding to the X-rays that are generated by the X-ray tube 111. The high-voltage generation device may be a transformer system or an inverter system. The X-ray high-voltage device 114 may be provided in the rotation frame 113 or may be provided in a fixed frame not illustrated in the drawing.

The control device 115 includes processing circuitry including a CPU and a drive mechanism such as a motor or an actuator. The control device 115 receives an input signal from an input interface 142 and controls operations of the gantry 110 and the bed 130. For example, the control device 115 performs control on rotation of the rotation frame 113, a tilt of the gantry 110, and operations of the bed 130. For example, the control device 115 causes the rotation frame 113 to rotate about an axis parallel to the X-axis direction according to input inclination angle (tilt angle) information as the control of tilting the gantry 110. Note that the control device 115 may be provided in the gantry 110 or may be provided in the console 140.

The wedge 116 is an X-ray filter for adjusting the dosage of X-rays applied from the X-ray tube 111. Specifically, the wedge 116 is an X-ray filter that attenuates X-rays that are applied from the X-ray tube 111 such that the X-rays applied from the X-ray tube 111 to the subject P have a predetermined distribution. For example, the wedge 116 is a wedge filter or a bow-tie filter and is manufactured by processing aluminum, or the like, such that the wedge 116 has a given target angle and a given thickness.

The collimator 117 includes a lead plate for narrowing the area of radiation with X-rays having been transmitted through the wedge 116 and forms a slit according to a combination of a plurality of lead plates, or the like. The collimator 117 is sometimes referred to as an X-ray diaphragm. FIG. 1 illustrates the case where the wedge 116 is arranged between the X-ray tube 111 and the collimator 117; however, the collimator 117 may be arranged between the X-ray tube 111 and the wedge 116. In this case, the wedge 116 transmits X-rays that are applied from the X-ray tube 111 and whose area of radiation is restricted by the collimator 117 and attenuates the X-rays.

The DAS 118 acquires signals of X-rays that are detected by the respective detection elements that the X-ray detector 112 includes. For example, the DAS 118 includes an A/D converter that performs an amplification processing on electric signals that are output from the respective detection elements and thus converts the electric signals into digital signals and generates detection data. The DAS 118 is enabled with, for example, a processor such as an application specific integrated circuit (ASIC).

The DAS 118 may be a simultaneous acquisition system or may be a sequential acquisition system. The DAS 118 that is a simultaneous acquisition system is provided for each detection element. The DAS 118 that is a simultaneous acquisition system reads electric charge at the same time when the electric charge is accumulated in the corresponding detection element and then generates detection data. The DAS 118 that is a sequential acquisition system is provided for each set of a plurality of detection elements (detection element group). The DAS 118 that is a sequential acquisition system sequentially reads the charge accumulated in each of the detection elements, thereby generating detection data.

The data that is generated by the DAS 118 is transmitted from a transmitter with a light emitting diode (LED) that is provided in the rotation frame 113 by optical communication to a receiver with a photodiode that is provided in a non-rotation part of the gantry 110 (for example, the fixed frame, or the like, of which illustration in FIG. 1 is omitted) and is transferred to the console 140. The non-rotation part is, for example, the fixed frame that supports the rotation frame 113 rotatably. A method of transmitting data from the rotation frame 113 to the gantry 110 is not limited to optical communication, and any non-contact data transmission system may be employed or contact data transmission system may be employed.

The bed 130 is a device on which the subject P to be scanned is laid and that moves the subject P and includes a base 131, a bed drive device 132, the tabletop 133, and a support frame 134. The base 131 is a casing that supports the support frame 134 movably in the vertical direction. The bed drive device 132 is a drive mechanism that moves the tabletop 133 on which the subject P is laid in a longitudinal direction of the tabletop 133 and includes a motor and an actuator. The tabletop 133 that is provided on a top surface of the support frame 134 is a board on which the subject P is laid. The bed drive device 132 may move, in addition to the tabletop 133, the support frame 134 in the longitudinal direction of the tabletop 133.

The console 140 includes a communication interface 141, the input interface 142, a display 143, a memory 144, and processing circuitry 145. The console 140 is described as one independent of the gantry 110; however, the console 140 or part of each component of the console 140 may be included in the gantry 110.

The communication interface 141, the input interface 142, the display 143, and the memory 144 can be configured similarly as the communication interface 21, the input interface 22, the display 23, and the memory 24 and therefore detailed description thereof will be omitted. For example, under the control of the processing circuitry 145, the display 143 displays the model M2 at step S103 and the installation positions at step S102 together with the organ model M1.

The processing circuitry 145 executes a controlling function 145a, an obtaining function 145b, an estimating function 145c, and an outputting function 145d, thereby controlling operations of the entire X-ray CT apparatus 100. The controlling function 145a is an example of a controller. The obtaining function 145b is an example of an obtaining unit. The estimating function 145c is an example of an estimator. The outputting function 145d is an example of an output unit.

For example, the processing circuitry 145 reads a program corresponding to the controlling function 145a from the memory 144 and executes the program, thereby functioning as the controlling function 145a. Similarly, the processing circuitry 145 can function as the obtaining function 145b, the estimating function 145c, and the outputting function 145d.

The controlling function 145a images the subject P and acquires X-ray CT images. For example, the controlling function 145a controls the X-ray high-voltage device 114, thereby supplying a high voltage to the X-ray tube 111. Accordingly, the X-ray tube 111 generates X-rays to be applied to the subject P. The controlling function 145a controls the bed drive device 132, thereby moving the subject P into an imaging port of the gantry 110. The controlling function 145a adjusts the position of the wedge 116 and the degree of opening and position of the collimator 117, thereby controlling the distribution of the X-rays to be applied to the subject P. The controlling function 145a controls the control device 115, thereby rotating the rotation unit. While the controlling function 145a is executing scanning, the DAS 118 acquires electric signals that are output from the respective detection elements in the X-ray detector 112 and generates detection data.

The controlling function 145a performs pre-processing on the detection data that is output from the DAS 118. For example, the controlling function 145a performs pre-processing, such as logarithmic transformation and offset correction processing, sensitivity correction processing between channels and beam hardening correction processing, on the detection data that is output from the DAS 118. The data on which the pre-processing has been performed is also referred to as raw data. The detection data on which the pre-processing has not been performed and the raw data on which the pre-processing has been performed are collectively referred to also as projection data.

The controlling function 145a executes reconstruction processing based on the projection data and generates an X-ray CT image (volume data). A method of reconstruction is not particularly limited and, for example, any method such as a filter correction back projection method or a successive approximation reconstruction method.

The obtaining function 145b is the same function as the obtaining function 25a. For example, the obtaining function 145b obtains the organ model M1 representing an anatomical structure based on the X-ray CT image.

The estimating function 145c is the same function as the estimating function 25b. For example, based on the organ model M1 and the installation positions in which the treatment device is installed, the estimating function 145c estimates feature values after treatment.

The outputting function 145d is the same function as the outputting function 25c. For example, based on a condition on display corresponding to the feature value, the outputting function 145d causes the information representing the treatment device and the installation positions to be displayed together with the organ model M1.

In the X-ray CT apparatus 100 illustrated in FIG. 9, each of the processing functions is stored in a form of a computer-executable program in the memory 144. For example, the processing circuitry 145 is a processor that reads the program from the memory 144 and executes the program, thereby implementing the function corresponding to each of the programs. In other words, the processing circuitry 145 having read the program has the function corresponding to the read program.

FIG. 9 illustrates that the single processing circuitry 145 implements the controlling function 145a, the obtaining function 145b, the estimating function 145c and the outputting function 145d; however, a plurality of independent processors may be combined to configure the processing circuitry 145 and the respective processors may execute the programs, thereby implementing the functions. Each of the processing functions that the processing circuitry 145 includes may be distributed or integrated to or into a single or a plurality of processing circuits as appropriate and be implemented. FIG. 9 illustrates the case where the programs corresponding to the respective processing functions are stored in the single memory 144; however, a configuration in which the programs corresponding to the respective functions are distributed to a plurality of memories and are stored in the memories and each memory reads and executes each program may be employed.

The processing circuitry 145 may implement the functions using a processor of an external device that is connected via the network NW. For example, the processing circuitry 145 reads the program corresponding to each of the functions from the memory 144 and executes the program and uses a server group (cloud) that is connected to the X-ray CT apparatus 100 via the network NW as calculation resources, thereby implementing each of the functions illustrated in FIG. 1.

The word “processor” used in the description given above refers to, for example, a CPU, a graphics processing unit (GPU), or a circuit such as an application specific integrated circuit (ASIC) or a programmable logic device (for example, a simple programmable logic device (SPLD), a complex programmable logic device (CPLD), or a field programmable gate array (FPGA)). The processor reads the program saved in the memory and executes the memory, thereby implementing the function.

FIG. 1 illustrates that the single memory 24 stores the programs corresponding to the respective processing functions. Embodiments however are not limited to this. For example, a plurality of the memories 24 may be arranged in a distributed manner and the processing circuitry 25 may be configured to read a corresponding program from the individual memory 24. As for FIG. 9, similarly, a plurality of the memories 144 may be arranged in a distributed manner and the processing circuitry 145 may be configured to read a corresponding program from the individual memory 144. Instead of saving the programs in the memory, the programs may be directly installed in a circuit of a processor. In this case, the processor reads the programs that are installed in the circuit and executes the programs, thereby implementing the functions.

Each of the components of each device according to the above-described embodiments is a functional idea and thus need not necessarily be configured physically as unillustrated in the drawings. In other words, specific modes of distribution and integration of the devices are not limited to those illustrated in the drawings, and all or part of the devices may be configured in a distributed or integrated manner functionally or physically in any unit according to various types of load and the usage. Furthermore, all or any part of the processing functions implemented by the respective devices may be implemented by a CPU and programs that are analyzed and executed by the CPU or may be implemented as hardware using a wired logic.

It is possible to implement the method described in the above-described embodiments by executing a program that is prepared in advance with a computer such as a personal computer or a work station. The program can be distributed via a network such as the Internet. The program may be recorded in a computer-readable and non-transient recording medium, such as a hard disk, a flexible disk (FD), a CD-ROM, a MO or a DVD, and may be read from the recording medium by the computer and thus be executed.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An X-ray CT apparatus comprising processing circuitry configured to:
image a subject and acquire X-ray CT images;
based on the X-ray CT images, obtain an organ model representing an anatomical structure;
based on the organ model and a plurality of installation positions in which a treatment device is installed, estimate feature values after treatment; and
based on a condition on display corresponding to the feature value, cause information representing the treatment device and the installation positions to be displayed together with the organ model.

2. A medical information processing apparatus comprising processing circuitry configured to:
obtain an organ model representing an anatomical structure;
based on the organ model and a plurality of installation positions in which a treatment device is installed, estimate feature values after treatment; and
based on a condition on display corresponding to the feature value, cause information representing the treatment device and the installation positions to be displayed together with the organ model.

3. The medical information processing apparatus according to claim 2, wherein the processing circuitry is configured to set the condition on display of the information representing the treatment device based on the feature value.

4. The medical information processing apparatus according to claim 2, wherein the processing circuitry is configured to set the installation positions with respect to each of a plurality of treatment conditions, and
cause a plurality of sets of the information representing the treatment device and the installation positions to be displayed with respect to the treatment conditions, respectively.

5. The medical information processing apparatus according to claim 2, wherein the organ model is a model representing a mitral valve, and
the installation positions are two positions that are set in an anterior leaflet and a posterior leaflet of the mitral valve, respectively.

6. The medical information processing apparatus according to claim 2, wherein the information representing the treatment device is a line segment connecting the installation positions.

7. he medical information processing apparatus according to claim 3, wherein the organ model is a model representing a mitral valve, and
the processing circuitry is configured to set the condition on display such that, the larger a postoperative regurgitant flow is, the more visibility lowers.

8. The medical information processing apparatus according to claim 2, wherein the organ model is a model representing a mitral valve, and
the processing circuitry is configured to set the installation positions with respect to each of a plurality of treatment conditions, and
set the condition on display of the information representing the treatment device based on the feature value and, based on the condition on display, cause a plurality of sets of the information representing the treatment device and the installation positions according to the treatment conditions, respectively.

9. The medical information processing apparatus according to claim 8, wherein the processing circuitry is configured to cause the information representing the treatment device and the installation positions that correspond to a give number of or a given ratio of the feature values of larger postoperative regurgitant flows among the feature values that are estimated with respect to the treatment conditions, respectively, to be displayed.

10. The medical information processing apparatus according to claim 8, wherein the processing circuitry is configured to cause the information representing the treatment device and the installation positions that correspond to a given number of or a given ratio of the feature values of smaller postoperative orifice areas among the feature values that are estimated with respect to the treatment conditions, respectively, to be displayed.

11. The medical information processing apparatus according to claim 2, wherein the processing circuitry is configured to set the condition on display according to at least any one of a size of the treatment device and an angle of installation of the treatment device.

12. The medical information processing apparatus according to claim 11, wherein the processing circuitry is configured to set the condition on display such that, the larger the size is, or the larger the angle of installation is, the more the information representing the treatment device is enhanced.

13. The medical information processing apparatus according to claim 6, wherein the processing circuitry is configured to set, as the condition on display, at least one of a thickness of the line segment, a line style, a line color, a permeation rate, presence or absence of end points, and a mark style of end points.

14. The medical information processing apparatus according to claim 7, wherein the processing circuitry is configured to estimate a regurgitant flow after treatment in the mitral valve as the feature value, and
set the condition on display such that, the larger the regurgitant flow is, the more the information representing the treatment device is in a mode of display different from enhanced display.

15. The medical information processing apparatus according to claim 7, wherein the processing circuitry is configured to estimate a prognostic state corresponding to the installation position in which the treatment device is installed, and
set the condition on display such that, the better the prognostic state is, the more the information representing the treatment device is displayed in an enhanced manner.

16. The medical information processing apparatus according to claim 2, wherein the treatment device is a clip that is installed in an anterior leaflet and a posterior leaflet of a mitral valve.

17. The medical information processing apparatus according to claim 2, wherein, when a plurality of the treatment devices are set, the processing circuitry is configured to estimate feature values after treatment based on the organ model and a plurality of installation positions corresponding to the treatment devices.

18. The medical information processing apparatus according to claim 17, wherein, based on a condition on display corresponding to the feature value, the processing circuitry is configured to cause information representing the treatment devices and the installation positions corresponding to the treatment devices to be displayed together with the organ model.

19. A method comprising:
obtaining an organ model representing an anatomical structure,
based on the organ model and a plurality of installation positions in which a treatment device is installed, estimating feature values after treatment, and
based on a condition on display corresponding to the feature value, causing information representing the treatment device and the installation positions to be displayed together with the organ model.

20. A non-transitory computer readable medium comprising instructions that cause a computer to execute:
obtaining an organ model representing an anatomical structure,
based on the organ model and a plurality of installation positions in which a treatment device is installed, estimating feature values after treatment, and
based on a condition on display corresponding to the feature value, causing information representing the treatment device and the installation positions to be displayed together with the organ model.
